(19) United States
(12) Patent Application Publication
(10) Pub. No.: US 2025/0001102 A1
(43) Pub. Date: Jan. 2, 2025

(54) MEDICATION ADJUSTMENT DEVICE AND NEBULIZER

(71) Applicant: HCMed Innovations Co., LTD., Taipei City (TW)

(72) Inventors: HSIN-YI PAI, New Taipei City (TW); YU-CHUNG HSU, New Taipei City (TW)

(21) Appl. No.: 18/759,277

(22) Filed: Jun. 28, 2024

Related U.S. Application Data
(60) Provisional application No. 63/524,202, filed on Jun. 29, 2023.

(30) Foreign Application Priority Data
Jun. 21, 2024 (CN) 202410805423.4

(51) Int. Cl. A61M 11/00

(52) U.S. Cl. CPC A61M 11/00

(57) ABSTRACT

A nebulizer includes a medication adjustment member and a nebulizing module. The medication adjustment device includes a liquid reservoir and a medication adjustment member. The liquid reservoir includes a first opening and a second opening. The liquid reservoir includes an accommodating cavity, and the first opening communicates with the second opening through the accommodating cavity. The medication adjustment member is detachably disposed on the first opening and located in the accommodating cavity. The medication adjustment member includes a third opening and an adjustment opening. The medication adjustment member includes an adjustment chamber therein. The third opening communicates with the adjustment opening, and the adjustment opening communicates with the second opening. The nebulizing module is disposed inside the liquid reservoir. The nebulizing module is located at a connection position between the second opening and the accommodating cavity.

### CROSS-REFERENCE TO RELATED PATENT APPLICATION

This application claims the benefit of priority to the U.S. Provisional Patent Application Ser. No. 63/524,202 filed on Jun. 29, 2023, which application is incorporated herein by reference in its entirety.

This application claims the benefit of priority to China Patent Application No. 202410805423.4, filed on Jun. 21, 2024 in People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a medication adjustment device and a nebulizer, and more particularly to a medication adjustment device and nebulizer capable of controlling the amount of medicinal liquid.

### BACKGROUND OF THE DISCLOSURE

Nebulizers for medical use mainly deliver drugs through the respiratory system. The nebulizers nebulize the medicinal liquid into small particles with a specific particle size and deliver the small particles from the user's mouth and nose into the respiratory system of the user's body through breathing for treatment purposes.

A reservoir inside the existing nebulizer contains a fixed amount of medicinal liquid, and the nebulizer cannot control the specific amount of medicinal liquid to be nebulized in the reservoir. Furthermore, while packaging medicinal liquid, a fixed amount of the medicinal liquid is contained in a medicine package. When a user pours the medicinal liquid in the medicine package into the nebulizer, the amount of medicinal liquid in the medicine package is not necessarily equal to the amount of the medicinal liquid required by the user. For example, if the amount of the medicine package in the medicine package is 10 ml, the amount of the medicinal liquid required by the user may only be 5 ml. Therefore, it is difficult for the user to adjust the amount of the medicinal liquid poured into the nebulizer by himself.

On the other hand, during treatment with a nebulizer, the activated nebulizer continues to nebulize until the medicinal liquid in the reservoir is exhausted. If the amount of medicinal liquid required for treatment is lower than the amount of liquid in the medicine package, it is difficult for the user to evaluate when to manually turn off the nebulizer, which can result in an excessive amount of the medicinal liquid being inhaled, thereby affecting the therapeutic effect.

### SUMMARY OF THE DISCLOSURE

In response to the above-referenced technical inadequacies, the present disclosure provides a medication adjustment device and a nebulizer.

In order to solve the above-mentioned problems, one of the technical aspects adopted by the present disclosure is to provide a medication adjustment device, which includes a liquid reservoir and a medication adjustment member. The liquid reservoir includes a first opening and a second opening. The liquid reservoir includes an accommodating cavity, and the first opening communicates with the second opening through the accommodating cavity. The medication adjustment member is detachably disposed on the first opening and located in the accommodating cavity. The medication adjustment member includes a third opening and an adjustment opening. The medication adjustment member includes an adjustment chamber therein. The third opening communicates with the adjustment opening, and the adjustment opening communicates with the second opening.

In order to solve the above-mentioned problems, another one of the technical aspects adopted by the present disclosure is to provide a nebulizer, which includes a medication adjustment device and a nebulizing module. The medication adjustment device includes a liquid reservoir and a medication adjustment member. The liquid reservoir includes a first opening and a second opening. The liquid reservoir includes an accommodating cavity, and the first opening communicates with the second opening through the accommodating cavity. The medication adjustment member is detachably disposed on the first opening and located in the accommodating cavity. The medication adjustment member includes a third opening and an adjustment opening. The medication adjustment member includes an adjustment chamber therein. The third opening communicates with the adjustment opening, and the adjustment opening communicates with the second opening. The nebulizing module is disposed inside the liquid reservoir and located at a connection position between the second opening and the accommodating cavity.

Therefore, in the medication adjustment device and the nebulizer provided by the present disclosure, the medication adjustment member is disposed inside the liquid reservoir of the nebulizer, and the adjustment chamber in the medication adjustment member is used to accommodate redundant amount of the medicinal liquid in the medicine package, so as to ensure that the amount of the medicinal liquid flowing into the liquid reservoir is exactly the amount of the medicinal liquid required by the user.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a medication adjustment device according to a first embodiment of the present disclosure;

FIG. 2 is a schematic exploded view of the medication adjustment device according to the first embodiment of the present disclosure;

FIG. 3 is a schematic view of a liquid reservoir according to the first embodiment of the present disclosure;

FIG. 4 is a schematic cross-sectional view of a liquid reservoir and a medication adjustment member according to the first embodiment of the present disclosure;

FIG. 5 is a schematic cross-sectional view of the medication adjustment device according to the first embodiment of the present disclosure;

FIG. 6 is a schematic exploded view of a medication adjustment device according to a second embodiment of the present disclosure;

FIG. 7 is a schematic cross-sectional view of a liquid reservoir and a medication adjustment member according to the second embodiment of the present disclosure;

FIG. 8 is a schematic cross-sectional view of the medication adjustment device according to the second embodiment of the present disclosure; and

FIG. 9 is a schematic view of a nebulizer according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

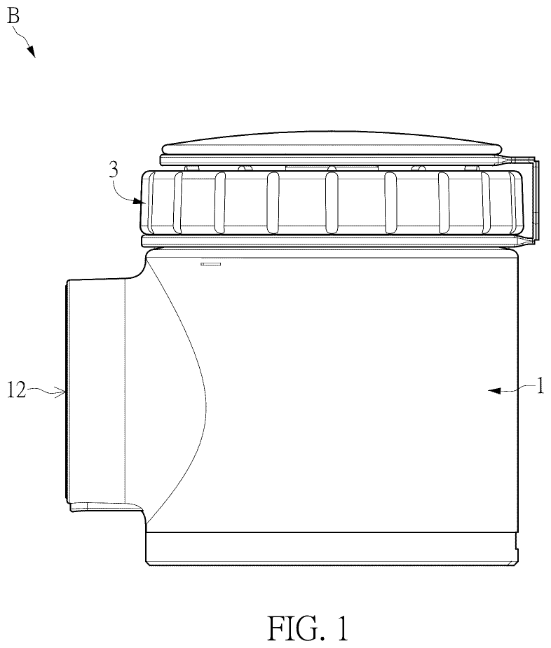

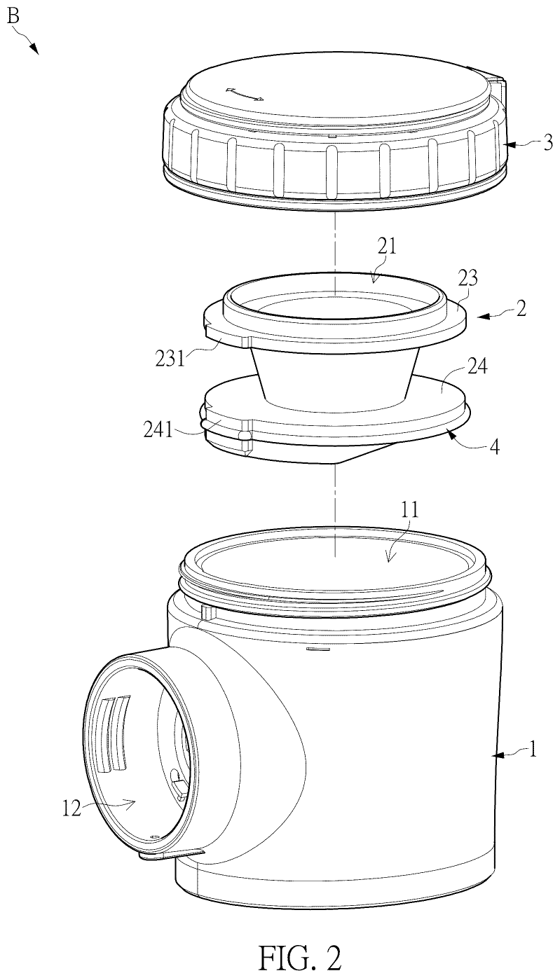

Reference is made to FIG. 1 and FIG. 2. A first embodiment of the present disclosure provides a medication adjustment device B that can be applied to a nebulizer. The medication adjustment device B includes a liquid reservoir 1, a medication adjustment member 2, and a cover 3. The liquid reservoir 1 includes a first opening 11 and a second opening 12. The medication adjustment member 2 is detachably disposed inside the liquid reservoir 1. The cover 3 is disposed on the liquid reservoir 1 and covers the first opening 11 of the liquid reservoir 1.

Reference is made to FIG. 2 to FIG. 4. The liquid reservoir 1 includes an accommodating cavity C1 therein, and the first opening 11 communicates with the second opening 12 through the accommodating cavity C1. The medication adjustment member 2 is disposed in the accommodating cavity C1. The medication adjustment member 2 includes a third opening 21 and an adjustment opening 22. The third opening 21 communicates with the adjustment opening 22, and the adjustment opening 22 communicates with the second opening 12 of the liquid reservoir 1. When the medication adjustment member 2 is disposed inside the reservoir 1, the third opening 21 of the medication adjustment member 2 overlaps with the first opening 11 of the reservoir 1. Therefore, the cover 3 is disposed on the liquid reservoir 1 to cover the first opening 11 as well as the third opening 21. In addition, the medication adjustment member 2 includes an adjustment chamber C2 therein.

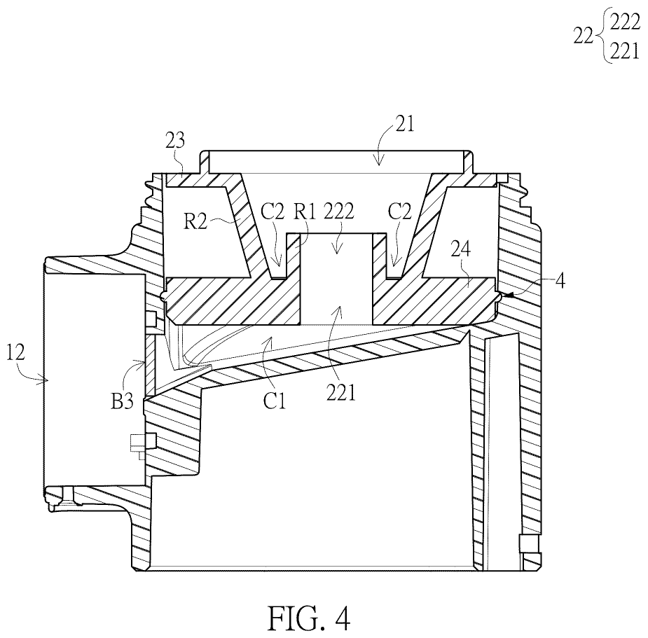

Reference is made to FIG. 4. In the first embodiment, the adjustment opening 22 is a cylindrical opening that includes a first port 221 and a second port 222. The first port 221 penetrates a bottom of the medication adjustment member 2, and the second port 222 extends toward the third opening 21, such that the adjustment opening 22 has a height from the bottom of the medication adjustment member 2. Moreover, a direction in which the adjustment opening 22 extends is perpendicular to a direction in which the second opening 12 extends. The adjustment chamber C2 is formed between a first peripheral wall R1 of the adjustment opening 22 and a second peripheral wall R2 of the medication adjustment member 2.

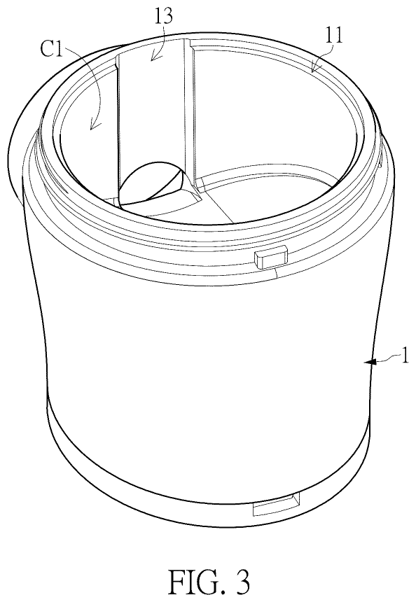

Reference is made to FIG. 2 and FIG. 3. The medication adjustment member 2 further includes a first annular portion 23 and a second annular portion 24. The first annular portion 23 is disposed around an outer periphery of the third opening 21. The second annular portion 24 is located below the first annular portion 23 and extends outward along a radial direction of the adjustment opening 22 to partially cover an area of the accommodating cavity C1. The second annular portion 24 is disposed around an outer periphery of the adjustment opening 22. The first annular portion 23 includes a first limiting protrusion portion 231. The second annular portion 24 includes a second limiting protrusion portion 241. An inner wall of the liquid reservoir 1 includes a limiting groove 13. Contour shapes of the first limiting protrusion portion 231 and the second limiting protrusion portion 241 correspond to a contour shape of the limiting groove 13. When the medication adjustment member 2 is disposed in the accommodating cavity C1, the first limiting protrusion portion 231 and the second limiting protrusion portion 241 are engaged (e.g., snapped) into the limiting groove 13, such that the medicine adjustment member 2 is disposed inside the liquid reservoir 1 in a fixed orientation.

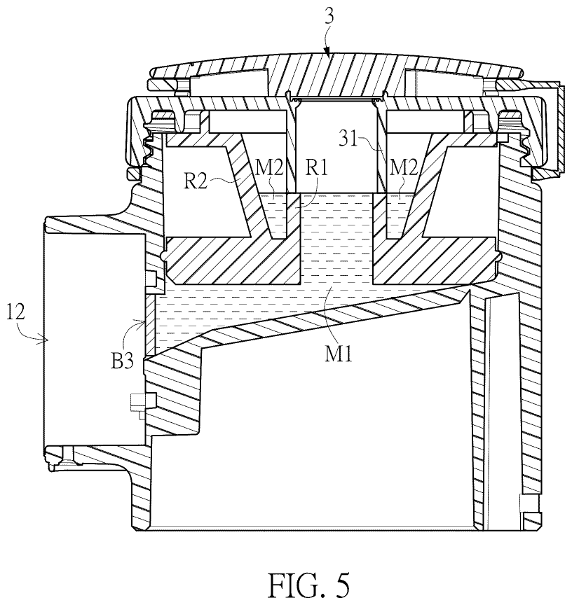

Reference is made to FIG. 2, FIG. 4, and FIG. 5. The medication adjustment device B further includes a sealing member 4. The sealing member 4 is surroundingly disposed outside the second annular portion 24. When the medicine adjustment member 2 is disposed in the accommodating cavity C1, the sealing member 4 is engaged (e.g., snapped) between the second annular portion 24 and the inner wall of the liquid reservoir 1. The sealing member 4 can be, for example, an elastic sealing ring (i.e., an O-ring). Alternatively, in other embodiments, the sealing member 4 can be a rib that is integrally formed with the second annular portion 24.

Reference is made to FIG. 5. The cover 3 includes an annular partition wall 31. When the cover 3 is disposed on the liquid reservoir 1, the annular partition wall 31 abuts against the first peripheral wall R1 of the adjustment opening 22 to close the second port 222. Since a volume of the adjustment chamber C2 of the medicine adjustment member 2 can be determined at the beginning of the design, designers can estimate the redundant amount of the medicinal liquid in the medicine package based on the amount of the medicinal liquid in the medicine package and the required amount of the medicinal liquid for the user, and accordingly design the volume in the adjustment chamber C2 to be equal to the volume of the redundant medicinal liquid.

As shown in FIG. 5, the amount of the medicinal liquid M1 in the accommodating cavity C1 and the adjustment opening 22 is the amount of medicinal liquid expected to be used (i.e., the required amount of medicinal liquid for the user). The amount of the medicinal liquid M2 in the adjustment chamber C2 is the residual amount of medicinal liquid (i.e., the redundant amount of the medicinal liquid). When the user pours the medicinal liquid in the medicine package into the liquid reservoir 1 with the medicine adjustment member 2 inside, the medicinal liquid enters through the third opening 21 and flows into the accommodating cavity C1 through the adjustment opening 22, and fills a space in the accommodating cavity C1 and the adjustment opening 22. Furthermore, when the amount of medicinal liquid is excessive, the redundant amount of the medicinal liquid overflows from the adjustment opening 22 and flows into the adjustment chamber C2. After that, the cover 3 covers the third opening 21, and the annular partition wall 31 abuts against the first peripheral wall R1 of the adjustment opening 22 to block the adjustment opening 22 from the adjustment chamber C2, which prevents the redundant amount of medicinal liquid in the adjustment chamber C2 from penetrating the accommodating cavity C1 while the nebulizer is shaken or tilted, and prevents the expected medicinal liquid in the accommodating cavity C1 from penetrating the adjustment chamber C2. In addition, through the arrangement of the sealing member 4, the medicinal liquid in the accommodating cavity C1 can be further prevented from leaking from a boundary of the second annular portion 24.

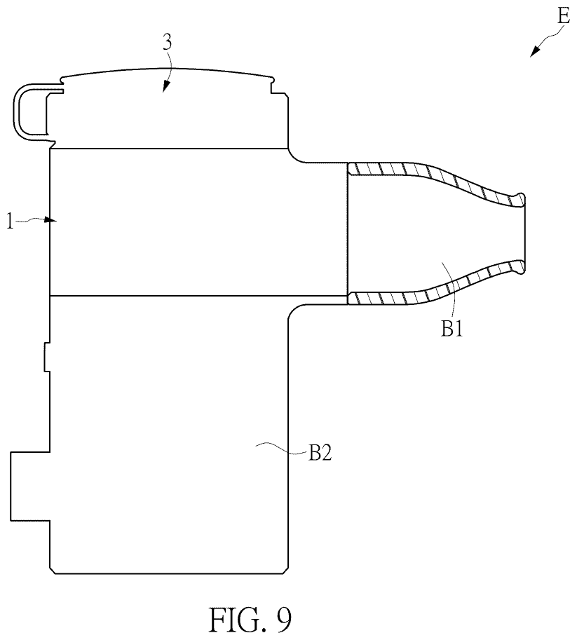

Reference is made to FIG. 5 and FIG. 9. The present disclosure also provides a nebulizer E, which includes the medication adjustment device B, and a nozzle assembly B1, a main body B2, and a nebulizing module B3 that are disposed on the medication adjustment device B. Specifically, the nozzle assembly B1 is disposed on the second opening 12 of the liquid reservoir 1, and the liquid reservoir 1 is disposed on the main body B2. The nebulizing module B3 is disposed inside the liquid reservoir 1 and is located at a connection position between the second opening 12 and the accommodating cavity C1. Therefore, the medicinal liquid expected to be used in the accommodating cavity C1 can be nebulized through the nebulizing module B3 to generate aerosol, and then enter the respiratory system of the user's body through the user's mouth and nose to achieve treatment purposes.

### Second Embodiment

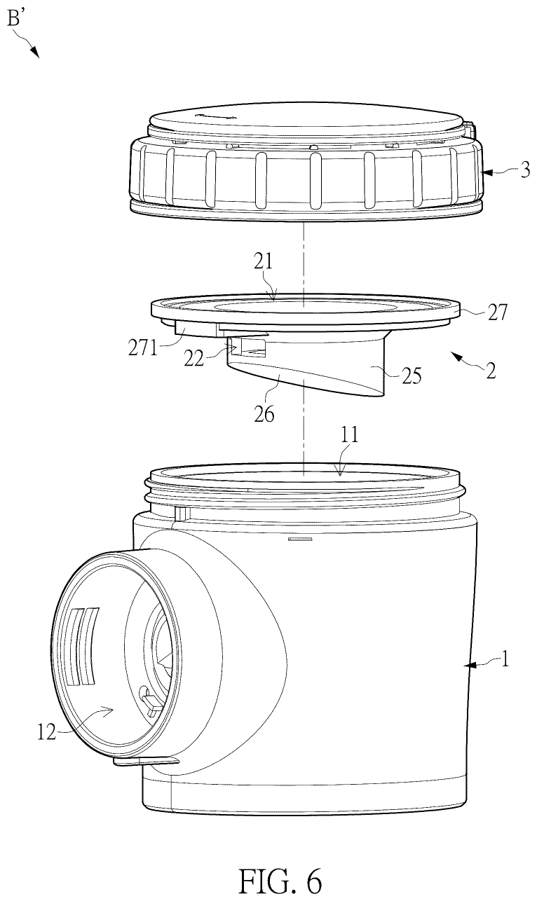

Referring to FIG. 6 to FIG. 8, a second embodiment of the present disclosure provides a medication adjustment device B′ that can be applied to a nebulizer E. The medication adjustment device B′ of the second embodiment is similar to the structure of the first embodiment, and the similarities (e.g., the liquid reservoir 1 and the cover 3) will not be reiterated herein. The main difference between the second embodiment and the first embodiment is the structure of the medication adjustment member 2.

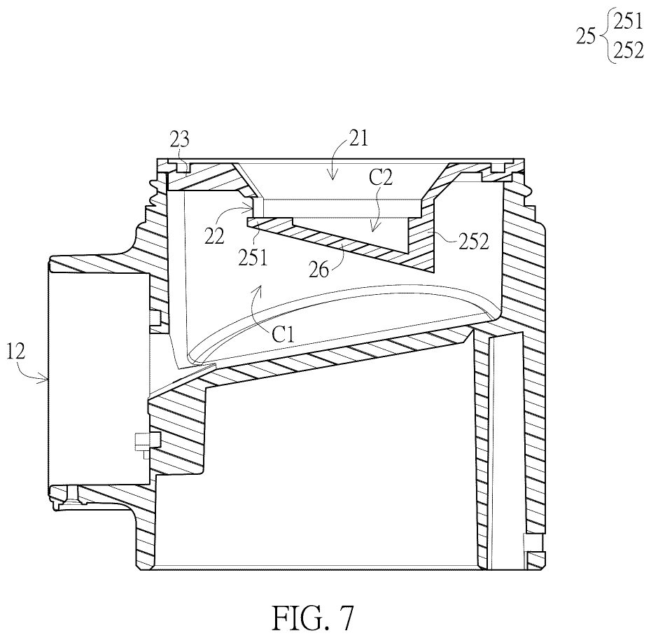

Specifically, in the second embodiment, the medication adjustment member 2 includes a third opening 21 and an adjustment opening 22, and the medication adjustment further includes an adjustment chamber C2 therein. The third opening 21 communicates with the adjustment opening 22, and the adjustment opening 22 communicates with the second opening 12. As shown in FIGS. 6 and 7, the medication adjustment member 2 is a funnel-shaped structure, which includes a side wall 25 and a bottom wall 26. The side wall 25 has a first side 251 and a second side 252 that are opposite to each other. The adjustment opening 22 is formed on the first side 251, and the adjustment opening 22 is positioned directly opposite to the second opening 12; that is, a direction in which the adjustment opening 22 extends is parallel to a direction in which the second opening 12 extends. The bottom wall 26 slopes downward from the first side 251 to the second side 252, and the bottom wall 26 and the side wall 25 jointly forms the adjustment chamber C2.

As shown in FIG. 6, the medication adjustment member 2 includes a supporting portion 27. The supporting portion 27 is disposed around an outer periphery of the third opening 21. The medication adjustment member 2 is detachably disposed in the first opening 11 and located in the accommodating cavity C1. Furthermore, the supporting portion 27 is engaged (e.g., snapped) on the edge of the first opening 11 and seals the first opening 11. The supporting portion 27 includes a limiting protrusion portion 271. The limiting protrusion portion 271 is located directly opposite to the adjustment opening 22. An inner wall of the liquid reservoir 1 includes a limiting groove 13 (in FIG. 3), and a contour shape of the limiting protrusion portion 271 corresponds to a contour shape of the limiting groove 13. When the medication adjustment member 2 is disposed in the accommodating cavity C1 of the liquid reservoir 1, the limiting protrusion portion 271 is disposed in the limiting groove 13, such that the medicine adjustment member 2 is disposed inside the liquid reservoir 1 in a fixed orientation.

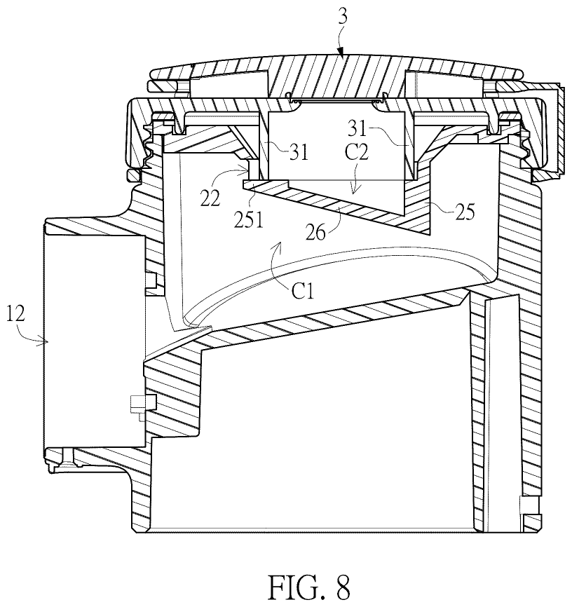

As shown in FIG. 8, when the cover 3 is disposed on the liquid reservoir 1, the annular partition wall 31 of the cover 3 abuts against the bottom of the liquid reservoir 1. Specifically, the annular partition wall 31 abuts against an upper edge of the side wall 25 of the liquid reservoir 1 to block the adjustment opening 22 from the adjustment chamber C2. When the user pours the medical liquid in the medicine package into the liquid reservoir 1 with the medicine adjustment member 2 inside, the medicinal liquid (not shown in FIG. 8) enters through the third opening 21 and flows into the accommodating cavity C1 through the adjustment opening 22, and fills a space in the accommodating cavity C1. An amount of the medicinal liquid that exceeds the amount to be adjusted by the medicine adjustment member 2 overflows from the regulating opening 22 and flows back to the adjustment chamber C2. After that, the cover 3 covers the third opening 21, and the annular partition wall 31 abuts against the side wall 25 to block the adjustment opening 22 from the adjustment chamber C2, which prevents the redundant amount of medicinal liquid in the adjustment chamber C2 from penetrating the accommodating cavity C1 while the nebulizer is shaken or tilted, and prevents the expected medicinal liquid in the accommodating cavity C1 from penetrating the adjustment chamber C2.

### Beneficial Effects of the Embodiments

In the medication adjustment device B, B′ and the nebulizer E provided by the present disclosure, the medication adjustment member 2 is disposed inside the liquid reservoir 1 of the nebulizer E, and the adjustment chamber C2 in the medication adjustment member 2 is used to accommodate a redundant amount of the medicinal liquid in the medicine package, so as to ensure that the amount of the medicinal liquid flowing into the liquid reservoir 1 is exactly the amount of the medicinal liquid required by the user. Moreover, in the medication adjustment device B, B′ provided by the present disclosure, the annular partition wall 31 blocks the adjustment opening 22 from the adjustment chamber C2 to prevent the redundant amount of medicinal liquid in the adjustment chamber C2 from penetrating the accommodating cavity C1 while the nebulizer is shaken or tilted, and prevents the expected medicinal liquid in the accommodating cavity C1 from penetrating the adjustment chamber C2.

Furthermore, since the medication adjustment member 2 and the liquid reservoir 1 are detachable components, the medication adjustment member 2 (e.g., the medication adjustment member 2 in FIG. 5 or FIG. 8) in the medication adjustment device B, B′ can be appropriately selected base on different users, such as adults and children, to accurately control the amount of the medicinal liquid and avoid overdose. In addition, the user can change the required amount of the medicinal liquid by replacing the medicine adjusting member 2 with one of a different volume. Therefore, the structure of the nebulizer E does not need to be significantly modified due to different amounts of the medicinal liquid to meet the needs of various medicine packages of different sizes.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A medication adjustment device, comprising:
a liquid reservoir including a first opening and a second opening, wherein the liquid reservoir includes an accommodating cavity, and the first opening communicates with the second opening through the accommodating cavity; and
a medication adjustment member detachably disposed on the first opening and located in the accommodating cavity, wherein the medication adjustment member includes a third opening and an adjustment opening, the medication adjustment member includes an adjustment chamber therein, the third opening communicates with the adjustment opening, and the adjustment opening communicates with the second opening.

2. The medication adjustment device according to claim 1, wherein the adjustment opening is a cylindrical opening that includes a first port and a second port, the first port penetrates a bottom of the medication adjustment member, and the second port extends toward the third opening.

3. The medication adjustment device according to claim 2, wherein the adjustment chamber is formed between a first peripheral wall of the adjustment opening and a second peripheral wall of the medication adjustment member.

4. The medication adjustment device according to claim 2, wherein the medication adjustment member further includes a first annular portion and a second annular portion, the second annular portion is located below the first annular portion, the first annular portion is disposed around an outer periphery of the third opening, and the second annular portion is disposed around an outer periphery of the adjustment opening.

5. The medication adjustment device according to claim 4, further comprising a sealing member, wherein the sealing member is surroundingly disposed outside the second annular portion.

6. The medication adjustment device according to claim 4, wherein the first annular portion includes a first limiting protrusion portion, the second annular portion includes a second limiting protrusion portion, an inner wall of the liquid reservoir includes a limiting groove, and contours of the first limiting protrusion portion and the second limiting protrusion portion correspond in shape to a contour of the limiting groove; wherein, when the medication adjustment member is disposed in the accommodating cavity, the first limiting protrusion portion and the second limiting protrusion portion are disposed in the limiting groove.

7. The medication adjustment device according to claim 2, further comprising a cover, wherein the cover is disposed on the liquid reservoir and covers the first opening and the third opening.

8. The medication adjustment device according to claim 7, wherein the cover includes an annular partition wall; wherein, when the cover is disposed on the liquid reservoir, the annular partition wall abuts against a first peripheral wall of the adjustment opening to block the adjustment opening from the adjustment chamber.

9. The medication adjustment device according to claim 1, wherein the medication adjustment member further includes a side wall and a bottom wall, the side wall has a first side and a second side that are opposite to each other, the adjustment opening is formed on the first side, and the bottom wall is tilted downward from the first side to the second side.

10. The medication adjustment device according to claim 9, the side wall and the bottom wall jointly form the adjustment chamber.

11. The medication adjustment device according to claim 9, wherein the medication adjustment member includes a supporting portion, the supporting portion is disposed around an outer periphery of the third opening, the supporting portion abuts against an edge of the first opening, and the adjustment opening is located directly opposite to the second opening.

12. The medication adjustment device according to claim 11, wherein the supporting portion includes a limiting protrusion portion, the limiting protrusion portion is located directly opposite to the adjustment opening, an inner wall of the liquid reservoir includes a limiting groove, and a contour of the limiting protrusion portion corresponds in shape to a contour of the limiting groove; wherein, when the medication adjustment member is disposed in the accommodating cavity, the limiting protrusion portion is disposed in the limiting groove.

13. The medication adjustment device according to claim 9, further comprising a cover, wherein the cover is disposed on the liquid reservoir and covers the first opening and the third opening.

14. The medication adjustment device according to claim 13, wherein the cover includes an annular partition wall; wherein, when the cover is disposed on the liquid reservoir, the annular partition wall abuts against a bottom of the adjustment opening to block the adjustment opening from the adjustment chamber.

15. A nebulizer, comprising:
the medication adjustment device as claimed in claim 1; and
a nebulizing module disposed inside the liquid reservoir, wherein the nebulizing module is located at a connection position between the second opening and the accommodating cavity.
